(19) United States
(12) Patent Application Publication
(10) Pub. No.: US 2012/0148637 A1
(43) Pub. Date: Jun. 14, 2012

(54) NANOPARTICULATE OLMESARTAN MEDOXOMIL COMPOSITIONS, PROCESS FOR THE PREPARATION THEREOF AND PHARMACEUTICAL COMPOSITIONS CONTAINING THEM

(76) Inventors: Genovéva Filipcsei, Bupapest (HU); Zsolt Ötvös, Csongrád (HU); Katalin Pongrácz, Budapest (HU); Ferenc Darvas, Budapest (HU)

(21) Appl. No.: 13/379,287

(22) PCT Filed: Jun. 18, 2010
(86) PCT No.: PCT/HU2010/000072

§ 371 Date: Feb. 22, 2012

(30) Foreign Application Priority Data
Jun. 19, 2009 (HU) P0900384

(51) Int. Cl. A61K 9/14; B32B 5/16; A61K 31/4178; A61P 9/12; B82Y 5/00

(52) U.S. Cl. 424/400; 514/382; 428/402; 977/773; 977/892; 977/906

(57) ABSTRACT

The present invention is directed to nanostructured (nanoparticulated) Olmesartan or its pharmaceutically acceptable ester, preferable Olmesartan Medoxomil, or co-crystal compositions, process for the preparation thereof and pharmaceutical compositions containing them. The nanoparticles of Olmesartan or its pharmaceutically acceptable ester, preferable Olmesartan Medoxomil, or co-crystal according to the invention have an average particle size of less than about 500 nm. Olmesartan Medoxomil is an angiotensin II receptor antagonist used to treat high blood pressure. The prodrug Olmesartan Medoxomil is marketed worldwide by Daiichi Sankyo, Ltd. and in the United States by Daiichi Sankyo, Inc.

### FIELD OF THE INVENTION

The present invention is directed to nanostructured (nanoparticulated) Olmesartan or its pharmaceutically acceptable ester, preferable Olmesartan Medoxomil, or co-crystal compositions, process for the preparation thereof and pharmaceutical compositions containing them.

The nanoparticles of Olmesartan or its pharmaceutically acceptable ester, preferable Olmesartan Medoxomil, or co-crystal according to the invention have an average particle size of less than about 500 nm. Olmesartan Medoxomil is an angiotensin II receptor antagonist used to treat high blood pressure. The prodrug Olmesartan Medoxomil is marketed worldwide by Daiichi Sankyo, Ltd. and in the United States by Daiichi Sankyo, Inc.

### BACKGROUND OF THE INVENTION

### A. Background Regarding to Nanoparticle Formation/Production

Nanoparticles development for Pharmaceutical Applications deals with emerging new technologies for developing customized solutions for drug delivery systems. The drug delivery systems should positively impact the rate of absorption, distribution, metabolism, and excretion of the drug or other related chemical substances in the body. In addition, the drug delivery system should allow the drug to bind to its target receptor and influence that receptor's signaling and activity. Drug delivery materials should be compatible, easy to bind with a particular drug, and able to degrade into fragments after use that are either metabolized or driven out via normal excretory routes.

A different approach is to produce the active ingredient (API) in nanoparticulate form.

Nanoparticle and microparticle compositions are described, for example, in US 20060281800, US 2007010592, WO 20081491553, WO 2008149160.

The API nanoparticles can be made using, for example, milling, homogenization, precipitation techniques, or supercritical fluid techniques, as is known in the art. Methods of making nanoparticulate compositions are also described in U.S. Pat. No. 5,718,388, U.S. Pat. No. 5,862,999, U.S. Pat. No. 5,665,331, U.S. Pat. No. 5,543,133, WO 2008149155 and EP 20060126293.

### B. Background Regarding Olmesartan Medoxomil

Olmesartan Medoxomil prodrug is hydrolyzed to Olmesartan during absorption from the gastrointestinal tract. Olmesartan is a selective AT1 subtype angiotensin II receptor antagonist.

Olmesartan Medoxomil is described chemically as 2,3-dihydroxy-2-butenyl 4-(1-hydroxy-1-methylethyl)-2-propyl-1-[p-(o-1H-tetrazol-5-ylphenyl)benzyl]imidazole-5-carboxylate, cyclic 2,3-carbonate.

Its empirical formula is C₂₉H₃₀N₆O₆ and its structural formula is:

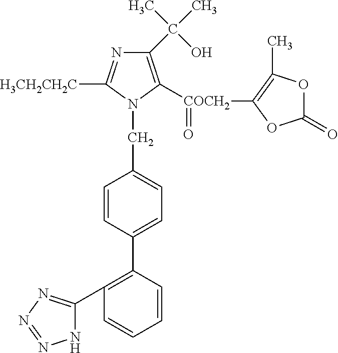

Olmesartan Medoxomil is a white to light yellowish-white powder or crystalline powder with a molecular weight of 558.59. It is practically insoluble in water and sparingly soluble in methanol. Benicar is available for oral use as film-coated tablets containing 5 mg, 20 mg, or mg of Olmesartan Medoxomil and the following inactive ingredients: hydroxypropylcellulose, lactose, low-substituted hydroxypropylcellulose, magnesium stearate, microcrystalline cellulose, talc, titanium dioxide, and (5 mg only) yellow iron oxide.

### Pharmacokinetics

### General

Olmesartan Medoxomil is rapidly and completely bioactivated by ester hydrolysis to Olmesartan during absorption from the gastrointestinal tract. Olmesartan appears to be eliminated in a biphasic manner with a terminal elimination half-life of approximately 13 hours. Olmesartan shows linear pharmacokinetics following single oral doses of up to 320 mg and multiple oral doses of up to 80 mg. Steady-state levels of Olmesartan are achieved within 3 to 5 days and no accumulation in plasma occurs with once-daily dosing.

The absolute bioavailability of Olmesartan is approximately 26%. After oral administration, the peak plasma concentration (Cₘₐₓ) of Olmesartan is reached after 1 to 2 hours. Food does not affect the bioavailability of Olmesartan.

### Metabolism and Excretion

Following the rapid and complete conversion of Olmesartan Medoxomil to Olmesartan during absorption, there is virtually no further metabolism of Olmesartan. Total plasma clearance of Olmesartan is 1.3 L/h, with a renal clearance of 0.6 L/h. Approximately 35% to 50% of the absorbed dose is recovered in urine while the remainder is eliminated in feces via the bile.

### Distribution

Olmesartan is highly bound to plasma proteins (99%) and does not penetrate red blood cells. The protein binding is constant at plasma Olmesartan concentrations well above the range achieved with recommended doses.

In rats, Olmesartan crossed the blood-brain barrier poorly, if at all. Olmesartan passed across the placental barrier in rats and was distributed to the fetus. Olmesartan was distributed to milk at low levels in rats.

### Side Effects

A very serious allergic reaction to this drug is unlikely, but unexplained skin rash, itching, unexplained swelling of the head or neck (including the face, lips, tongue, or throat), wheezing, difficulty breathing or swallowing, dizziness, lightheadedness, or fainting spells, decrease in the amount of urine produced can occur.

Because of the insolubility of Olmesartan and Olmesartan Medoxomil in water and the low bioavailability, there is a need in the art to enhance the lipophilicity/bioavailability/increase the absorption/reduce the side effect/decrease the dosage/faster onset of action, in order to overcome the problems associated with prior conventional Olmesartan Medoxomil formulations. Moreover, these problems can be solved by surface modification to decrease the first pass effect or modify the metabolism of Olmesartan Medoxomil. Beside the traditional formulation of Olmesartan Medoxomil, the transdermal application could decrease the time which is needed to reach the desired effect of Olmesartan Medoxomil. The present invention satisfies this need.

### DESCRIPTION OF THE INVENTION

The present invention describes the nanoparticulate Olmesartan, its pharmaceutically acceptable esters, preferable Olmesartan Medoxomil, and co-crystals composition with enhanced lipophilicity/bioavailability/increased absorption and dissolution rate/reduced side effect/decreased dosage/faster onset of action.

As exemplified in the examples below, not every combination of stabilizers will result in a stable nanoparticle formation. It was discovered, that stable, Olmesartan or its pharmaceutically acceptable ester, preferable Olmesartan Medoxomil, or co-crystal nanoparticles can be made by microfluidic based continuous flow method using selected stabilizers.

The invention comprises a stable nanoparticulate composition comprising:
(a) nanoparticulate Olmesartan or its pharmaceutically acceptable ester, preferable Olmesartan Medoxomil, or co-crystal having an average particle size of less than about 500 nm; and
(b) at least one stabilizer,

wherein the composition is prepared in a continuous flow reactor.

The composition of the invention is prepared in a continuous flow reactor, preferably in a microfluidic based continuous flow reactor.

In the composition of the invention the average particle size of Olmesartan or its pharmaceutically acceptable ester, preferable Olmesartan Medoxomil, or co-crystal is between 500 nm and 50 nm.

In the composition of the invention: (a) the Olmesartan or its pharmaceutically acceptable ester, preferable Olmesartan Medoxomil, or co-crystal is present in an amount selected from the group consisting of from about 99.5% to about 0.001%, from about 95% to about 0.1%, and from about 90% to about 0.5%, by weight, based on the total combined weight of the Olmesartan or its pharmaceutically acceptable ester, preferable Olmesartan Medoxomil, or co-crystal and at least one stabilizer, not including other excipients; (b) the stabilizer is present in an amount selected from the group consisting of from about 0.5% to about 99.999% by weight, from about 5.0% to about 99.9% by weight, and from about 10% to about 99.5% by weight, based on the total combined dry weight of the Olmesartan or its pharmaceutically acceptable ester, preferable Olmesartan Medoxomil, or co-crystal and at least one stabilizer, not including other excipients; or (c) a combination of (a) and (b).

In the composition of the invention the Olmesartan or its pharmaceutically acceptable ester, preferable Olmesartan Medoxomil, or co-crystal can be used in a crystalline phase, an amorphous phase, a semi-crystalline phase, a semi-amorphous phase, and co-crystal, and in mixtures thereof in any polymorph form.

For the preparation of the composition of the invention stabilizers include nonionic, anionic, cationic, ionic, and zwitterionic surfactants can be used. Combinations of more than one stabilizer can be used in the invention. Useful stabilizers which can be employed in the invention include, but are not limited to, known organic and inorganic pharmaceutical excipients. Such excipients include various polymers, low molecular weight oligomers, natural products, and surfactants.

Representative examples of stabilizers include hydroxypropyl methylcellulose, hydroxypropylcellulose, poly(vinylpyrrolidone), sodium lauryl sulfate, gelatin, dextran, stearic acid, glycerol monostearate, cetostearyl alcohol, sorbitan esters, polyoxyethylene castor oil derivatives, polyoxyethylene sorbitan fatty acid esters (e.g., the commercially available Tween® products such as e.g., Tween® 20 and Tween® 80 (ICI Speciality Chemicals)); polyethylene glycols (e.g., Carbowax® 3550 and 934 (Union Carbide), poly(meth)acrylate-based polymers and copolymers (Eudargit®), acetic acid ethenyl ester polymer with 1-ethenyl-2-pyrrolidinone (PVP/VA copolymers), sodium dodecyl benzene sulfonate, tocopheryl polyethylene glycol succinates, polyethoxylated castor oils and its derivateives, polyoxyethylene stearates, methylcellulose, hydroxyethylcellulose, cellulose acetate phthalate, polyvinyl alcohol (PVA), 4-(1,1,3,3-tetramethylbutyl)-phenol polymer with ethylene oxide and formaldehyde (also known as tyloxapol, superione, and triton), poloxamers (e.g., Pluronics, which are block copolymers of ethylene oxide and propylene oxide); poloxamines (e.g., Tetronic, also known as Poloxamine, which is a tetrafunctional block copolymer derived from sequential addition of propylene oxide and ethylene oxide to ethylenediamine (BASF Wyandotte Corporation, Parsippany, N.J.); PEG-phospholipid, PEG-cholesterol, PEG-cholesterol derivative, PEG-vitamin A, PEG-vitamin E, lysozyme, poly(2-ethyl-2-oxazoline), poly(methyl vinyl ether), random copolymers of vinyl pyrrolidone and vinyl acetate, such as Plasdone S630, and the like.

Examples of useful ionic stabilizers include, but are not limited to polymers, biopolymers, polysaccharides, cellulosics, alginates, phospholipids, and nonpolymeric compounds, such as zwitterionic stabilizers, poly-n-methylpyridinium, anthryul pyridinium chloride, cationic phospholipids, chitosan, polylysine, polyvinylimidazole, polybrene, polymethylmethacrylate trimethylammoniumbromide bromide (PMMTMABr), benzalkonium chloride, hexadecyltrimethylammonium bromide, hexyldesyltrimethylammonium bromide (HDMAB), and poly(vinylpyrrolidone)-2-dimethylaminoethyl methacrylate dimethyl sulfate.

Advantages of the composition of the invention include, but are not limited to: (1) smaller tablet or other solid dosage form size and beneficial transdermal/topical application; (2) lower doses of drug required to obtain the same pharmacological effect as compared to conventional forms of Olmesartan Medoxomil; (3) increased bioavailability as compared to conventional forms of Olmesartan Medoxomil; (4) improved pharmacokinetic profiles; (5) an increased rate of dissolution for Olmesartan or Olmesartan Medoxomil nanoparticles as compared to conventional forms of the same active compound; (6) modified metabolism of Olmesartan or Olmesartan Medoxomil nanoparticles.

For the preparation of the composition of the invention methods can be used comprising a continuous solvent-antisolvent precipitation using one or more stabilizers or a continuous chemical precipitation to form nanoparticles.

Another aspect of the invention is a process for the preparation of nanostructured Olmesartan or its pharmaceutically acceptable ester, preferable Olmesartan Medoxomil, or co-crystal, comprising precipitating nanostructured Olmesartan or its pharmaceutically acceptable ester, preferable Olmesartan Medoxomil, or co-crystal an appropriate solution of Olmesartan or its pharmaceutically acceptable ester, preferable Olmesartan Medoxomil with one or more stabilizers if desired in the presence of a pharmaceutically acceptable acid or base in a continuous flow reactor.

As a continuous flow reactor a microfluidic based continuous flow reactor may be used.

The microfluidics based continuous flow reactor used is described in the publication Microfluid Nanofluid DOI 10.1007/s10404-008-0257-9 by I. Hornyak, B. Borcsek and F. Darvas.

Preferably the process may be carried out by (1) dissolving Olmesartan or its pharmaceutically acceptable ester, preferable Olmesartan Medoxomil and optionally one or more stabilizers in a suitable solvent; (2) adding the formulation from step (1) to a solution comprising one or more stabilizers and if desired a pharmaceutically acceptable acid or base and (3) precipitating the formulation from step (2).

Alternatively the process may be carried out by (1) dissolving Olmesartan or its pharmaceutically acceptable ester, preferable Olmesartan Medoxomil and one or more stabilizers in a suitable solvent; (2) adding the formulation from step (1) to a solution optionally comprising one or more stabilizers and if desired a pharmaceutically acceptable acid or base and (3) precipitating the formulation from step (2).

As solvents (a) two different solvents miscible with each other, where is soluble only in one of them may be used, or (b) the same solvent may be used in the two steps, where Olmesartan or its pharmaceutically acceptable ester, preferable Olmesartan Medoxomil forms nanostructured particles, practically, with the restriction that the applied stabilizer(s) is soluble in the solvents used.

Such solvents may be dimethyl-sufloxide, methanol, ethanol, i-propanol, tetrahydrofuran, acetone, acetonitrile, and pyridine preferably.

The particle size of the nanoparticulate Olmesartan or its pharmaceutically acceptable ester, preferable Olmesartan Medoxomil, or co-crystal can be influenced by the solvents used, the flow rate and the Olmesartan or its pharmaceutically acceptable ester, preferable Olmesartan Medoxomil, or co-crystal—stabilizer ratio.

Another aspect of the invention is directed to the good/instantaneous redispersibility of solid nanosized form of Olmesartan or its pharmaceutically acceptable ester, preferable Olmesartan Medoxomil, or co-crystal in biologically relevant mediums, e.g.; physiological saline solution, pH=2.5HCl solution.

Another aspect of the invention is a pharmaceutical composition comprising a stable nanoparticulate Olmesartan or its pharmaceutically acceptable ester, preferable Olmesartan Medoxomil, or co-crystal composition of it according to the invention and optionally pharmaceutically acceptable auxiliary materials.

The pharmaceutical composition of the invention can be formulated: (a) for administration selected from the group consisting of oral, pulmonary, rectal, colonic, parenteral, intracisternal, intravaginal, intraperitoneal, ocular, otic, local, buccal, nasal, and topical administration; (b) into a dosage form selected from the group consisting of liquid dispersions, gels, aerosols, ointments, creams, lyophilized formulations, tablets, capsules; (c) into a dosage form selected from the group consisting of controlled release formulations, fast melt formulations, delayed release formulations, extended release formulations, pulsatile release formulations, and mixed immediate release and controlled release formulations; or (d) any combination of (a), (b), and (c).

The compositions can be formulated by adding different types of excipients for oral administration in solid, liquid, vaginal, rectal, local (powders, ointments or drops), or topical administration, and the like.

A preferred dosage form of the invention is a solid or liquid (cream/ointment) dosage form, although any pharmaceutically acceptable dosage form can be utilized.

For oral delivery into the human body nanoparticles can be also administered as their aqueous dispersion as the final dosage form. This is a way of delivery without further processing after nanoparticle formation. However, poor stability of the drug or polymer in an aqueous environment or poor taste of the drug may require the incorporation of the colloidal particles into solid dosage forms, i.e. into capsules and tablets.

Alternatively, the aqueous dispersion of the colloidal particles can be incorporated into the solid dosage form as a liquid, for example by granulation of suitable fillers with the colloidal dispersion to form a granulation. Such granules can subsequently be filled into capsules or be compressed into tablets. Alternatively, through layering of the dispersion onto e.g. sugar-pellets as carriers in a fluidized bed a solid form for nanoparticles can be. These ways of manufacturing tablet cores, or granules or pellets can potentially by followed by a coating step to reveal a film-coated tablet or film coated granules in a capsule as the final dosage form.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents, or vehicles including water, ethanol, polyols (propyleneglycol, polyethylene-glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants.

Solid dosage forms for oral administration include, but are not limited to, capsules, tablets, pills, powders, and granules. In such solid dosage forms, the active agent is admixed with at least one of the following: (a) one or more inert excipients (or carriers), such as sodium citrate or dicalcium phosphate; (b) fillers or extenders, such as starches, lactose, sucrose, glucose, mannitol, and silicic acid; (c) binders, such as carboxymethylcellulose, alignates, gelatin, poly(vinylpyrrolidone), sucrose, and acacia; (d) humectants, such as glycerol; (e) disintegrating agents, such as agar-agar, calcium carbonate, potato or tapioca starch, alginic acid, certain complex silicates, and sodium carbonate; (f) solution retarders, such as paraffin; (g) absorption accelerators, such as quaternary ammonium compounds; (h) wetting agents, such as cetyl alcohol and glycerol monostearate; (i) adsorbents, such as kaolin and bentonite; and (j) lubricants, such as talc, calcium stearate, magnesium stearate, solid polyethylene glycols, sodium lauryl sulfate, or mixtures thereof. For capsules, tablets, and pills, the dosage forms may also comprise buffering agents.

Liquid dosage forms for oral administration include pharmaceutically acceptable emulsions, solutions, suspensions, syrups, and elixirs. In addition to the Olmesartan or Olmesartan Medoxomil, the liquid dosage forms may comprise inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, oils, such as cottonseed oil, groundnut oil, corn germ oil, olive oil, castor oil, and sesame oil, glycerol, tetrahydrofurfuryl alcohol, polyethyleneglycols, fatty acid esters of sorbitan, or mixtures of these substances, and the like.

Besides such inert diluents, the composition can also include adjuvants, such as wetting agents, emulsifying and suspending agents, sweetening, flavoring, and perfuming agents.

The pharmaceutical compositions of the invention show enhanced lipophilicity/bioavailability/increased absorption and dissolution rate/reduced side effect/decreased dosage/faster onset of action as compared to conventional Olmesartan Medoxomil form.

The present invention is also directed to methods of treatment of hypertension using the novel Olmesartan and Olmesartan Medoxomil nanoparticles disclosed herein.

### A. Preferred Characteristics of the Olmesartan and Olmesartan Medoxomil Nanoparticles of the Invention

### 1. Increased Bioavailability

The nanoparticulate Olmesartan Medoxomil compositions of the invention are proposed to exhibit increased bioavailability and require smaller doses as compared to prior known, conventional Olmesartan Medoxomil formulations.

### Example 1

### In Vivo Pharmacokinetic Tests Male Sprague-Dawley Rats in Fasted Condition: Comparison of Reference Active Pharmaceutical Ingredient and Nanostructured Olmesartan Medoxomil

### Experimental Protocols

### Comparative In Vivo Pharmacokinetic Tests in Male Sprague-Dawley Rats in Fasted Condition

The single oral dose of reference Olmesartan Medoxomil was 20 mg/kg, and that of nanostructured Olmesartan Medoxomil formulation was 75.8 mg/kg which corresponds to 20 mg/kg active agent. Both test substances were administered via gastric tube in a dosing volume of 5 ml/kg. The vehicle of the test items was sterile 0.9% NaCl solution supplemented with 0.25% methylcellulose and the suspension was kept homogenous by continuous stirring during treatment in order to minimize the error resulting from the sedimentation.

### Animals

Male Wistar rats (purchased from Laboratory Animal Center, University of Szeged) were maintained on a standard pellet rodent diet (Bioplan Ltd, Isaszeg, Hungary) under temperature and light-controlled conditions with tap water available ad libitum. The acclimatization period was at least 4 days. Rats were randomized into groups of 6 and each group was used for blood sampling at different time period after Olmesartan Medoxomil treatment. All animals were fasting for 16 hours before oral treatment. Animals were anesthetized with halothane and blood has been withdrawn by cardiac puncture 15, 30, 45, 60, 120 and 360 minutes after Olmesartan Medoxomil treatment. Water was available immediately after treatment for all animals. Rats in the last group (sacrificed at 360 min) had access to standard rodent food 120 minutes after the treatment. Serum samples were prepared by centrifugation (7000 rμm, 10 min, 4° C.) of the clotted blood within 60 minutes and were stored at −20° C. till analysis.

### Sample Preparation

An aliquot of 200 μl serum was combined with 20 μl of internal standard working solution and 1.2 ml methanol for protein precipitation. The mixture was vortexed for 1 min and centrifuged at 12000 rμm for 10 min at 4° C. The supernatants were evaporated to dryness under a stream of nitrogen at 40° C. and reconstituted with 200 μl of water—methanol (50:50 v/v) and 20 μl was injected into the HPLC system. Serum Olmesartan concentrations were determined using a standard curve set up from Olmesartan prepared from Olmesartan Medoxomil by enzymatic hydrolysis.

### Statistical Analysis

Statistical analysis and graph drawing were carried out be GraphPad Prism 4.0 (GraphPad Software, San Diego, USA).

### Results

Both reference active pharmaceutical and nanostructured Olmesartan Medoxomil treatment resulted in a detectable serum Olmesartan concentration exhibiting a biphasic profile in the 15-360 min interval after the oral administration of 20 mg/kg test substance. The absorption of Olmesartan Medoxomil from the nanostructured formula was obviously faster and more complete than after the administration of reference substance. Following nanostructured Olmesartan Medoxomil treatment the maximal serum Olmesartan concentration (Cₘₐₓ) was 7.885±1.915 μg/ml, while for the reference preparation it was 1.802±0.400 μg/ml. Furthermore, Cₘₐₓ was observed at an earlier time point (tₘₐₓ=120 min and 60 min for the reference and the nanostructured formulation, respectively).

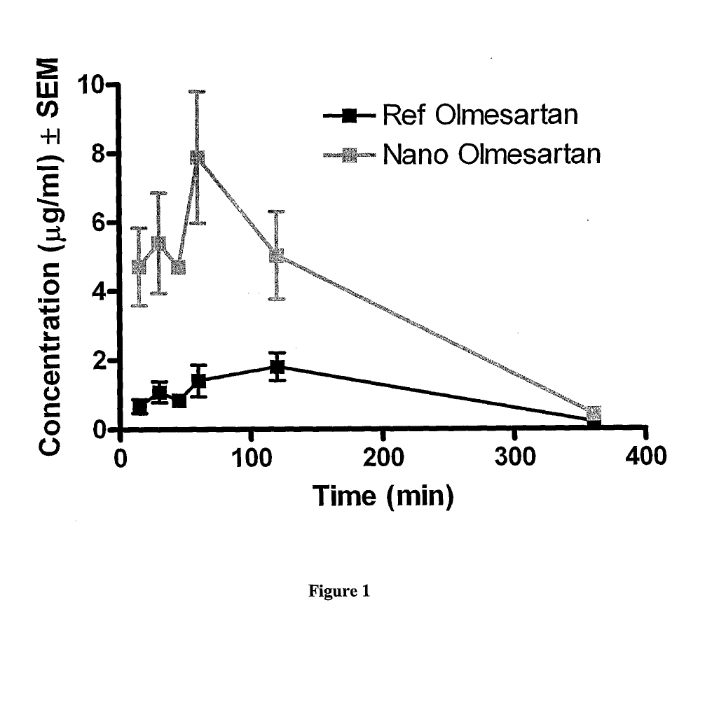

Area under the serum concentration curve between 15 and 360 min (AUC_{15-360 min}) has been calculated to characterize the extent of the absorption of the test items. AUC_{15-360 min} was 382.5 μg*min/ml and 1228.0 μg*min/ml for the reference compound and the nanostructured formula, respectively. The ratio of the two AUC values (AUC_{15-360 min (nanosized)}/AUC_{15-360 min (reference)}) was 3.4 (FIG. 1).

FIG. 1: Serum concentrations of Olmesartan after oral administration of 20 mg/kg nanostructured and reference test substance

### 2. Dissolution Profiles of the Nanoparticulate Olmesartan Medoxomil Compositions of the Invention

The nanoparticulate Olmesartan and Olmesartan Medoxomil compositions of the invention have increased solubility and dissolution profile due to the decreased particles size and unique nanostructured particle formation. Rapid dissolution of an administered active agent is preferable, as faster dissolution generally leads to faster onset of action and greater bioavailability.

### Example 2

### Experimental Protocol

### Determination of Solubility (C_{Max})

The solubility of nanostructured Olmesartan Medoxomil compared to the reference API was determined in distilled water by UV-VIS measurements (Agilent 8453) at 254 nm wavelength and room temperature. The redispersed sample was filtered by 0.20 μm disposable syringe filter. In order to check the nanoparticle presence in the solution, it was irradiated by red laser pointer operating at 670 nm wavelength. If no scattering was observed the filtration was successful, the solution did not contain nanoparticles.

### Dissolution Tests

Dissolution tests were performed by redispersing 9.5 mg nanostructured Olmesartan Medoxomil powder containing 2.5 mg Olmesartan Medoxomil and 2.5 mg reference Olmesartan Medoxomil in 10 mL distillate water. The suspension was stirred for 1, 5, 10, 15, 20, 30 and 60 minutes and then it was filtered by 0.2 μm disposable syringe filter. Olmesartan Medoxomil concentration was determined by UV-VIS spectrophotometer (Agilent 8453).

### Results

### Determination of Cₘₐₓ

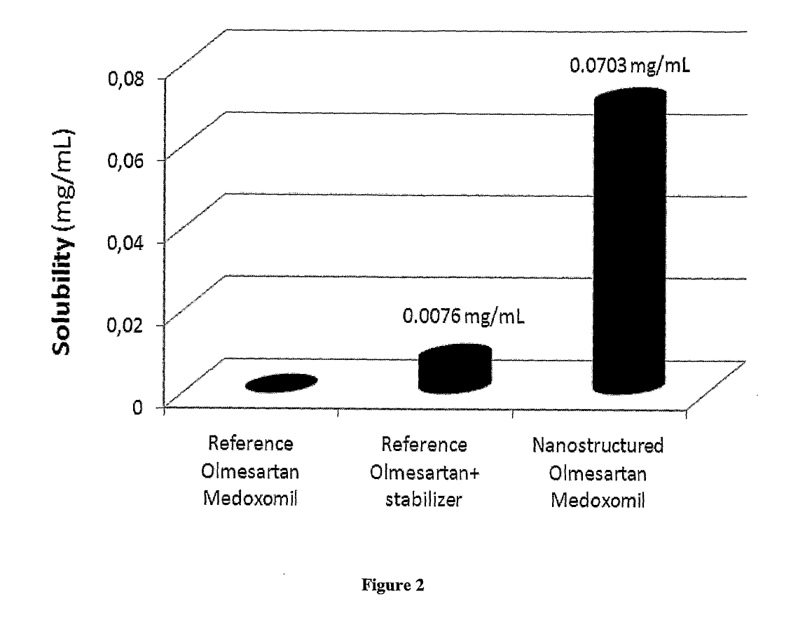

Redispersibility test was performed in order to determine the solubility of the nanostructured Olmesartan Medoxomil. The mean particle size of the redispersed nanostructured Olmesartan Medoxomil was 283 nm by intensity based average and 266 nm by numeric average. The d(90) values were 382 and 318 nm by intensity based and numeric average, respectively. The solubility of the nanostructured Olmesartan Medoxomil was 9.07 mg/mL which is 9 times higher than the solubility of Olmesartan Medoxomil in distilled water (FIG. 2).

FIG. 2: Solubility enhancement of Olmesartan Medoxomil

### Comparative Dissolution Test

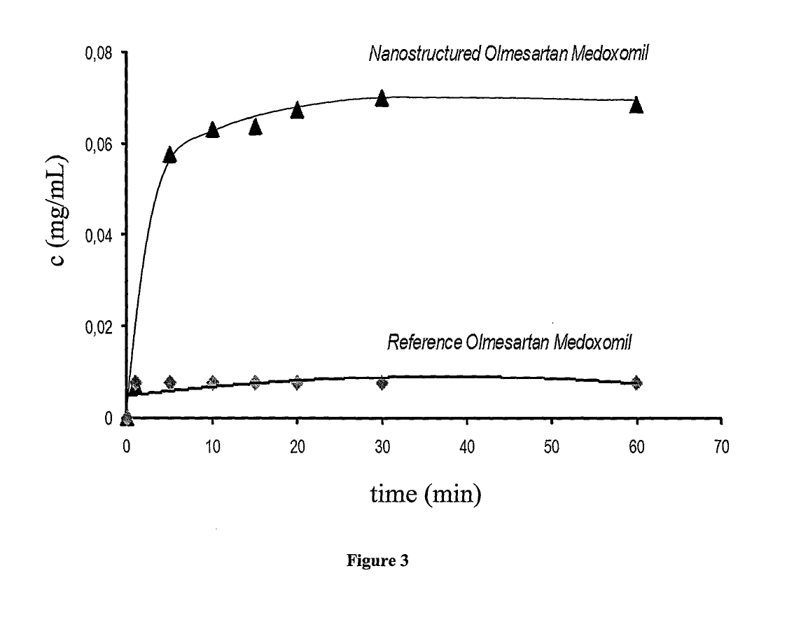

Due to the instantaneous redispersibility of nanostructured Olmesartan Medoxomil of example 6, more than 10% of the Olmesartan Medoxomil content of the composition dissolves immediately upon the redispersion. Within 10 minutes the solution containing the redispersed nanostructured particles reaches its saturated state, the dissolved Olmesartan Medoxomil content is 0.0703 mg/mL which is in a good correlation with the solubility of nanostructured Olmesartan Medoxomil (FIG. 3).

FIG. 3: Comparative dissolution test of reference Olmesartan Medoxomil and nanostructured Olmesartan Medoxomil

### 3. Increased Solubility by Amorphous/Partly Crystalline/Crystalline/Polymorph/Co-Crystal State of the Nanoparticulate Olmesartan Medoxomil Compositions of the Invention

The chemical stability of solid drugs is affected by the crystalline state of the drug. Many drug substances exhibit polymorphism. Each crystalline state has different chemical reactivity. The stability of drugs in their amorphous form is generally lower than that of drugs in their crystalline form, because of the higher free-energy level of the amorphous state.

Decreased chemical stability of solid drugs brought about by mechanical stresses such as grinding is to a change in crystalline state.

The chemical stability of solid drugs is also affected by the crystalline state of the drug through differences in surface area. For reaction that proceeds on the solid surface of drug, an increase in the surface area can increase the amount of drug participating in the reaction.

### Example 3

### Crystallographic Structure Determination

Stable partly crystalline, crystalline, polymorph or amorphous nanostructured Olmesartan Medoxomil compositions of the invention show significantly enhanced solubility due to its increased surface area when compared to a crystalline reference.

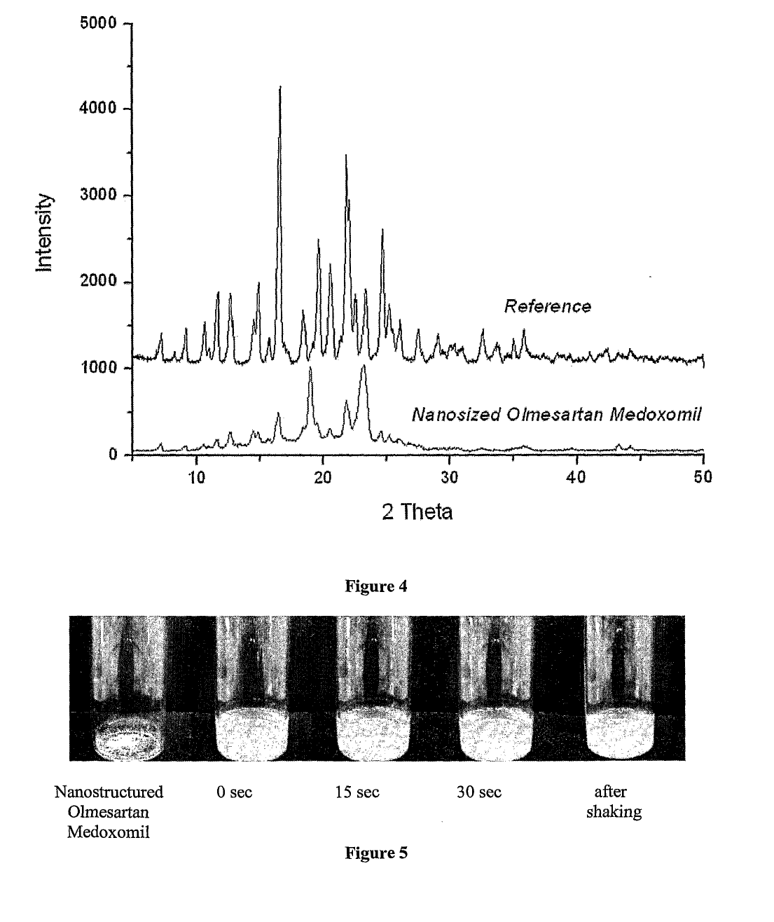

The structure of the Olmesartan Medoxomil nanoparticles prepared by continuous flow nano precipitation method was investigated by X-ray diffraction analysis (Philips PW1050/1870 RTG powder-diffractometer). The measurements showed that the nanostructured Olmesartan Medoxomil compositions are crystalline (See in FIG. 4). The characteristic reflections of the crystalline Olmesartan Medoxomil can be found on the XRD diffractogram of nanosized Olmesartan Medoxomil, but with lower intensity (FIG. 4).

FIG. 4: X-ray diffractograms of reference Olmesartan Medoxomil and Nanostructured Olmesartan Medoxomil Composition of the Invention

### 4. Redispersibility Profiles of the Nanoparticulate Olmesartan Medoxomil Compositions of the Invention

An additional feature of the nanoparticulate Olmesartan Medoxomil compositions of the present invention is that the dried nanoparticles stabilized by surfactant/(s)/polimer(s) can be redispersed instantaneously or using traditional redispersants such as mannitol, sucrose.

### Example 4

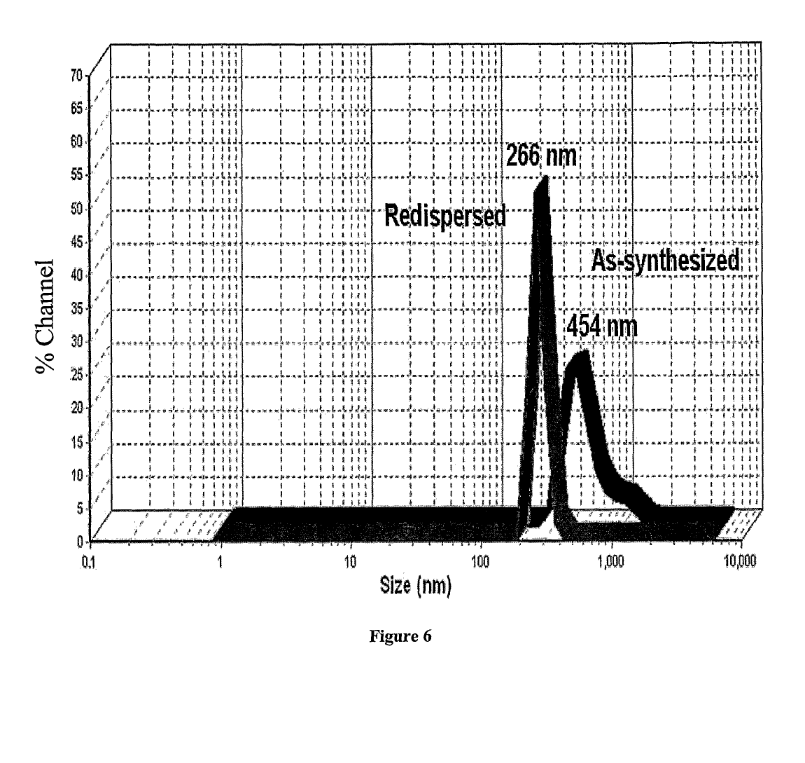

The redispersibility of nanostructured Olmesartan Medoxomil powder was performed by dispersing 5 mg nanosized Olmesartan Medoxomil powder in 5 mL distilled water. Following the distilled water addition the vial was gentle shaken by hand resulting in colloid dispersion of nanostructured Olmesartan Medoxomil particles as it is demonstrated in FIG. 5. The particle size and size distribution of the redispersed particles can be seen in FIG. 6.

FIG. 5: Instantaneous redispersibility of nanostructured Olmesartan Medoxomil in distilled water

FIG. 6: Size and size distribution of the Olmesartan Medoxomil nanoparticles before and after the redispersion

### 5. Enhanced Lipophilicity to Increase the Absorption and Permeability Profiles of the Nanoparticulate Olmesartan Medoxomil Compositions of the Invention

Due to the phospholipidic nature of cell membranes, a certain degree of lipophilicity is oftentimes a requirement for the drug compound, not only to be absorbed through the intestinal wall following oral administration but possibly also to exert its pharmacological action in the target tissue. (F. Kesisoglou et al./Advanced Drug Delivery Reviews 59 (2007) 631-644).

The lipophilicity of the Olmesartan and Olmesartan Medoxomil can be increased by using lipophilic stabilizer or/and stabilizers having lipophilic side groups on the polymeric backbone and/or amphiphil stabilizers during the precipitation. Due to the lipophilic nature or lipophilic side groups of the applied stabilizer, not only the lipophilicity, but the absorption and the permeability of the Olmesartan and Olmesartan Medoxomil nanoparticles of the present invention can be increased.

For example using Chitosan, it can increase the paracellular permeability of intestinal epithelia which attributed to the transmucosal absorption enhancement.

Most amphiphilic copolymers employed for drug delivery purposes contain either a polyester or a poly(amino acid)-derivative as the hydrophobic segment. Most of the polyethers of pharmaceutical interest belong to the poloxamer family, i.e. block-copolymers of polypropylene glycol and polyethylene glycol.

### 6. Faster Surface Wetting Profiles of the Nanoparticulate Olmesartan Medoxomil Compositions of the Invention

For the Olmesartan and Olmesartan Medoxomil to dissolve, its surface has first to be wetted by the surrounding fluid. The nanosized amorphous/crystalline/polymorph forms possess a chemically randomized surface which expresses hydrophobic and hydrophilic interactions due to the nature of the stabilizer/(s) and active pharmaceutical ingredient, which can lead to improved wettability. If the surface of the Olmesartan and Olmesartan Medoxomil nanoparticles of the invention is functionalized by hydrophilic groups/stabilizer(s), a higher degree of hydrophility causes faster surface wetting and faster dissolution compared to the original crystalline form. This advanced property of the Olmesartan and Olmesartan Medoxomil nanoparticles of the present invention is supported by the results of the redispersibility test (See in Figure in 5). Due to the bigger surface, the polymorph character of the nanoparticles and the hydrophilic groups of the stabilizer(s) (e.g.: poloxamer—Pluronic PE 10500) the surface wetting is faster than the crystalline form's.

### B. Compositions

The invention provides nanosized Olmesartan and Olmesartan Medoxomil nanostructured particle formations comprising at least one stabilizer to stabilize them sterically and/or electrostatically.

The stabilizers preferably are associated or interacted with the Olmesartan and Olmesartan Medoxomil, but do not chemically react with the Olmesartan and Olmesartan Medoxomil or themselves.

The nanoparticles of Olmesartan and Olmesartan Medoxomil of the invention can be formed by solvent-antisolvent precipitation methods using stabilizer(s). The stability of the prepared colloid solution of nanosized Olmesartan and Olmesartan Medoxomil can be increased by the combination of additional stabilizer(s) which can act as a second steric or electrostatic stabilizer. Moreover, using additional stabilizer the particle size of Olmesartan and Olmesartan Medoxomil of the invention can be decreased and controlled.

Particle Size of Olmesartan Medoxomil nanoparticles

The invention contains Olmesartan Medoxomil nanoparticles, which have an average particle size of less than about 500 nm as measured by dynamic light scattering method.

By “an average particle size of less than about 500 nm” it is meant that at least 50% of the Olmesartan Medoxomil nanoparticles have a particle size of less than the average, by number/intensity, i.e., less than about 500 nm, etc., when measured by the above-noted technique.

### Example 5

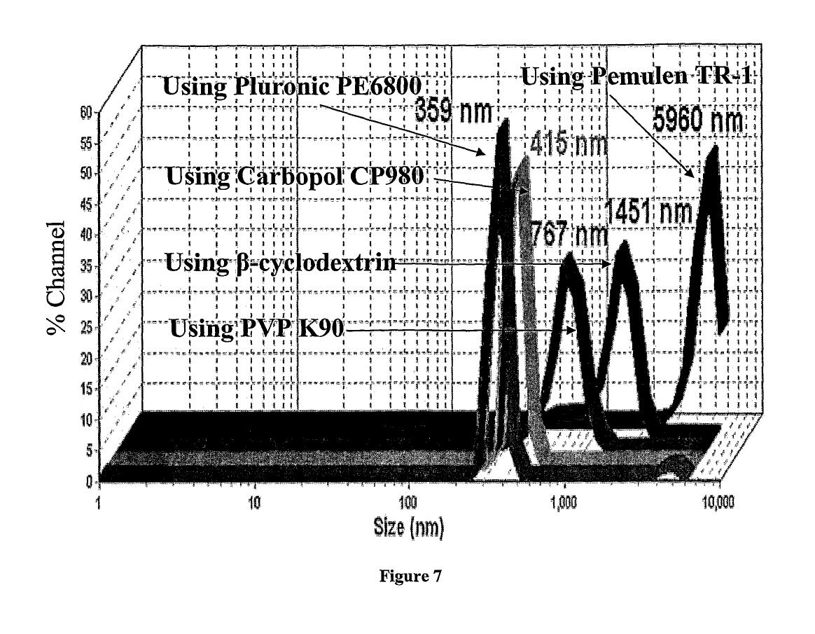

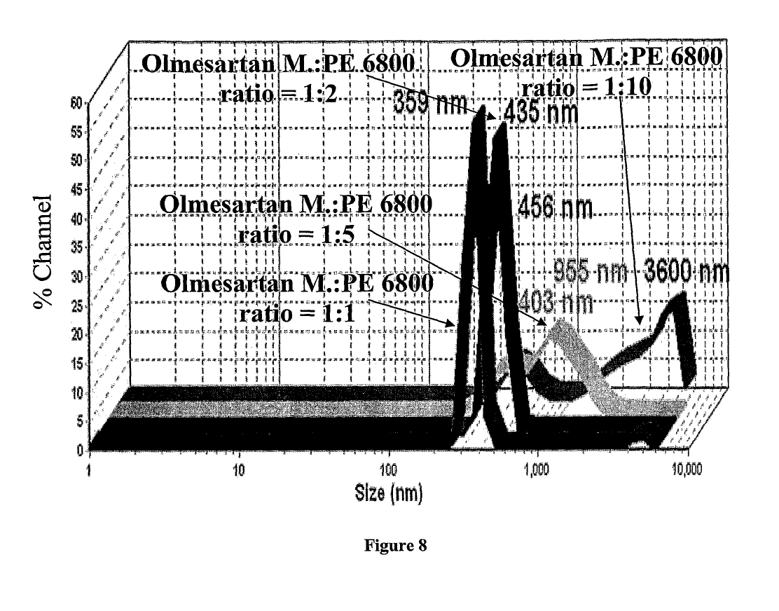

During the experiments Olmesartan Medoxomil nanoparticles were prepared in a microfluidic based continuous flow reactor. As a starting solution, 200 mg Olmesartan Medoxomil, 40 mg sodium dodecyl sulfate and 200 mg Pluronic 6800 dissolved in 100 mL DMSO was used. The prepared solution was passed into the reactor unit with 0.5 mL/min flow rate using a feeding unit. Meanwhile, using a second feeding unit, distilled water was passed into a mixing unit with 2 mL/min flow rate, where it was mixed with the solution containing Olmesartan Medoxomil coming from the first reactor unit. The nanoparticles are continuously produced at atmospheric pressure due to the chemical precipitation by water passed into the mixing unit. The produced colloidal solution driven through the second reactor unit getting to the dynamic light scattering unit (Nanotrac) integrated to the device, which can detect the particle size of the obtained nanoparticle continuously. The size of the nanoparticles can be controlled in wide range by changing the flow rates; pressure and the types of the stabilizers (see FIG. 7). The particles size and size distribution of the Olmesartan Medoxomil particles can be controlled by the amount the stabilizer(s) (Pluronic 6800) as it is show in FIG. 8. The particles size of the Olmesartan Medoxomil particle was 359 nm in the best case.

FIG. 7: Particle size and size distribution of Olmesartan Medoxomil nanoparticles using different stabilizers

FIG. 8: Effect of the Olmesartan Medoxomil—stabilizer ratio on the particle size and size distribution of Olmesartan Medoxomil nanoparticles

### Example 6

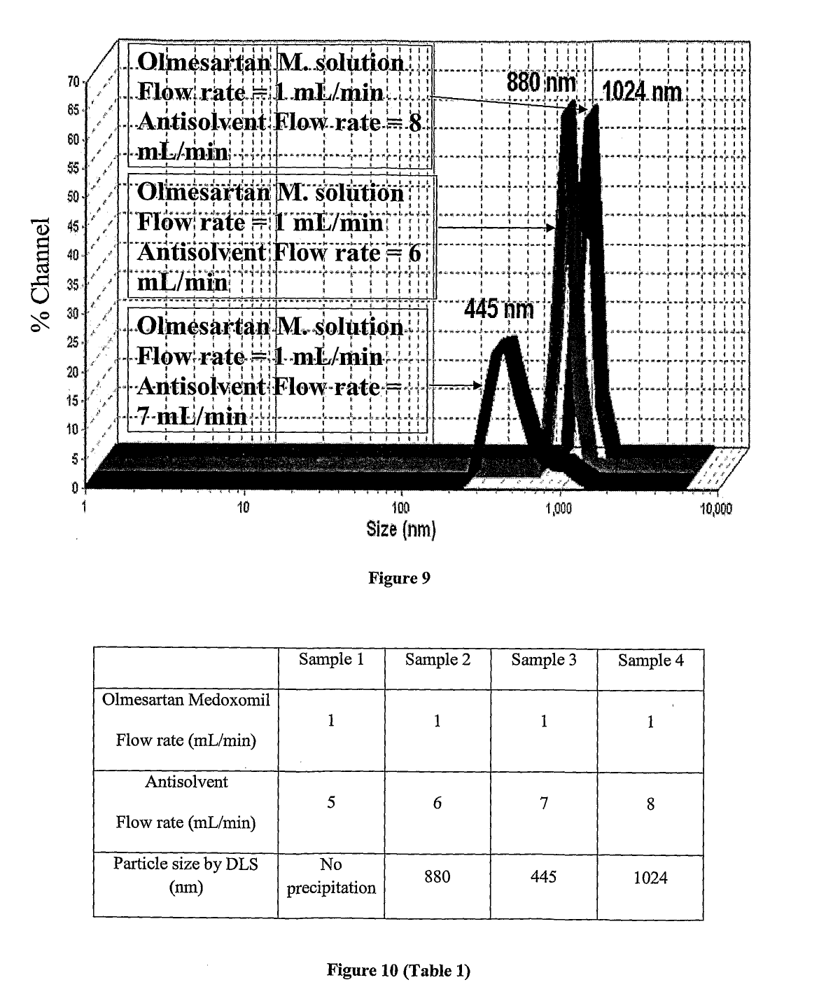

During the experiments Olmesartan Medoxomil nanoparticles were prepared in a microfluidic based continuous flow reactor. As a starting solution, 500 mg Olmesartan Medoxomil and 1 g Pluronic PE 10500 dissolved in 100 mL ethanol was used. The prepared solution was passed into the reactor unit with 1 mL/min flow rate using a feeding unit. Meanwhile, using a second feeding unit, 0.05% poly(ethyleneglycol) (PEG 2000) dissolved in distilled water was passed into a mixing unit with 7 mL/min flow rate, where it was mixed with the solution containing Olmesartan Medoxomil coming from the first reactor unit. The nanoparticles are continuously produced at atmospheric pressure due to the chemical precipitation by water passed into the mixing unit. The produced colloidal solution driven through the second reactor unit getting to the dynamic light scattering unit (Nanotrac) integrated to the device, which can detect the particle size of the obtained nanoparticle continuously. The size of the nanoparticles can be controlled in wide range by changing the flow rates and the types of the stabilizers. The particles size and size distribution of the Olmesartan Medoxomil particles can be controlled by the flow rates as it is show in FIG. 9. The particles size of the Olmesartan Medoxomil particle was 445 nm in the best case (see table 1).

FIG. 9: Effect of the flow rates on the particle size and size distribution of Olmesartan Medoxomil nanoparticles

Table 1: Effect on the flow rates on the particle size of Olmesartan Medoxomil

### Example 7

### Olmesartan Medoxomil Nanoparticles Loaded Cream Formulation

Preparing 100 ml gel containing Olmesartan Medoxomil nanoparticles 1.3 g Carbopol 971 was dissolved under vigorous stirring at room temperature in 100 mL Olmesartan Medoxomil colloidal solution as-synthesized by the method described in example 6.

1. Nanostructured Olmesartan, its pharmaceutically acceptable esters, preferably Olmesartan Medoxomil and co-crystals having an average particle size of less than about 500 nm.

2. Nanostructured Olmesartan, its pharmaceutically acceptable esters, preferably Olmesartan Medoxomil and co-crystals according to claim 1 wherein the average particle size is between 500 nm and 50 nm.

3. Nanostructured Olmesartan, according to claim 1 wherein the average particle size is between 500 nm and 50 nm.

4. Nanostructured Olmesartan esters, preferably Olmesartan Medoxomil according to claim 1 wherein the average particle size is between 500 nm and 50 nm.

5. A stable nanostructured composition comprising:
(a) nanostructured Olmesartan or its pharmaceutically acceptable ester, preferable Olmesartan Medoxomil, or co-crystal having an average particle size of less than about 500 nm; and
(b) at least one stabilizer.

6. A stable nanostructured composition according to claim 3, comprising:
(a) nanostructured Olmesartan or its pharmaceutically acceptable ester, preferable Olmesartan Medoxomil, or co-crystal having an average particle size of less than about 500 nm; and
(b) at least one stabilizer,
wherein the composition is prepared in a continuous flow reactor.

7. A stable nanostructured composition according to claim 4, comprising:
(a) nanostructured Olmesartan or its pharmaceutically acceptable ester, preferable Olmesartan Medoxomil, or co-crystal having an average particle size of less than about 500 nm; and
(b) at least one stabilizer,
wherein the composition is prepared in a microfluidic based continuous flow reactor.

8. A composition according to claims 6-8, comprising nanostructured Olmesartan having an average particle size between 500 nm and 50 nm.

9. A composition according to claims 6-8, comprising nanostructured Olmesartan Medoxomil having an average particle size between 500 nm and 50 nm.

10. The composition according to claims 6-10, wherein: (a) the Olmesartan or its pharmaceutically acceptable ester, preferable Olmesartan Medoxomil, or co-crystal is present in an amount selected from the group consisting of from about 99.5% to about 0.001%, from about 95% to about 0.1%, and from about 90% to about 0.5%, by weight, based on the total combined weight of the Olmesartan or its pharmaceutically acceptable ester, preferable Olmesartan Medoxomil, or co-crystal and at least one stabilizer, not including other excipients; (b) the stabilizer is present in an amount selected from the group consisting of from about 0.5% to about 99.999% by weight, from about 5.0% to about 99.9% by weight, and from about 10% to about 99.5% by weight, based on the total combined dry weight of the Olmesartan or its pharmaceutically acceptable ester, preferable Olmesartan Medoxomil, or co-crystal and at least one stabilizer, not including other excipients; or (c) a combination of (a) and (b).

11. The composition according to claims 6-12, wherein the Olmesartan or its pharmaceutically acceptable ester, preferable Olmesartan Medoxomil, or co-crystal is selected from the group consisting of a crystalline phase, an amorphous phase, a semi-crystalline phase, a semi-amorphous phase, a co-crystal and mixtures thereof in any polymorph form.

12. The composition of claims 1-12, comprising as stabilizers: hydroxypropyl methylcellulose, cellulose acetate phthalate, hydroxypropylcellulose, poly(vinylpyrrolidone), sodium lauryl sulfate, gelatin, dextran, stearic acid, glycerol monostearate, cetostearyl alcohol, sorbitan esters, polyoxyethylene castor oil derivatives, poly(meth)acrylate-based polymers and copolymers; acetic acid ethenyl ester polymer with 1-ethenyl-2-pyrrolidinone (PVP/VA copolymers), sodium dodecyl benzene sulfonate, tocopheryl polyethylene glycol succinates, polyethoxylated castor oils and its derivateives, polyoxyethylene sorbitan fatty acid esters; polyethylene glycols, polyoxyethylene stearates, methylcellulose, hydroxyethylcellulose, polyvinyl alcohol, 4-(1,1,3,3-tetramethylbutyl)-phenol polymer with ethylene oxide and formaldehyde, poloxamers; poloxamines, which is a tetrafunctional block copolymer derived from sequential addition of propylene oxide and ethylene oxide to ethylenediamine; PEG-phospholipid, PEG-cholesterol, PEG-cholesterol derivative, PEG-vitamin A, PEG-vitamin E, lysozyme, poly(2-ethyl-2-oxazoline), poly(methyl vinyl ether), random copolymers of vinyl pyrrolidone and vinyl acetate.

13. The composition of claims 1-13, comprising as additional stabilizer hydroxyl-propyl-cellulose derivatives, any other stabilizers, preferably combination of two stabilizers of claim 9 and/or lauryl trimethyl ammonium chloride, alkylbenzyl methyl ammonium chloride, alkylbenzyl dimethy lammonium bromide, benzyl trimethyl ammonium bromide, benzalkonium chloride, hexadecyltrimethylammonium bromide.

14. Process for the preparation of nanostructured Olmesartan or its pharmaceutically acceptable ester, preferable Olmesartan Medoxomil, or co-crystal according to claims 1 to 14, comprising precipitating nanostructured Olmesartan or its pharmaceutically acceptable ester, preferable Olmesartan Medoxomil, or co-crystal from an appropriate solution of Olmesartan or its pharmaceutically acceptable ester, preferable Olmesartan Medoxomil with one or more stabilizers if desired in the presence of a pharmaceutically acceptable acid or base in a continuous flow reactor.

15. Process according to claim 15. using as a continuous flow reactor a microfluidic based continuous flow reactor.

16. Process according to claims 15-16, comprising (1) dissolving Olmesartan or its pharmaceutically acceptable ester, preferable Olmesartan Medoxomil and optionally one or more stabilizers in a suitable solvent; (2) adding the formulation from step (1) to a solution comprising one or more stabilizers and if desired a pharmaceutically acceptable acid or base and (3) precipitating the formulation from step (2).

17. Process according to claims 15-16, comprising (1) dissolving Olmesartan or its pharmaceutically acceptable ester, preferable Olmesartan Medoxomil and one or more stabilizers in a suitable solvent; (2) adding the formulation from step (1) to a solution optionally comprising one or more stabilizers and if desired a pharmaceutically acceptable acid or base and (3) precipitating the formulation from step (2).

18. Process according to claims 15-18 comprising (a) using two different solvents miscible with each other, where Olmesartan or its pharmaceutically acceptable ester, preferable Olmesartan Medoxomil is soluble only in one of them, or (b) using the same solvent in the two steps, where Olmesartan or its pharmaceutically acceptable ester, preferable Olmesartan Medoxomil, or co-crystal forms nanostructured particles, practically, with the restriction that the applied stabilizer(s) is soluble in the solvents used.

19. A pharmaceutical composition comprising a nanostructured Olmesartan or its pharmaceutically acceptable ester, preferable Olmesartan Medoxomil, or co-crystal according to claims 1-19 and optionally pharmaceutically acceptable auxiliary materials.

20. The pharmaceutical composition of claim 20, wherein the composition is formulated: (a) for administration selected from the group consisting of oral, pulmonary, rectal, colonic, parenteral, intracisternal, intravaginal, intraperitoneal, ocular, otic, local, buccal, nasal, and topical administration; (b) into a dosage form selected from the group consisting of liquid dispersions, gels, aerosols, ointments, creams, lyophilized formulations, tablets, capsules; (c) into a dosage form selected from the group consisting of controlled release formulations, fast melt formulations, delayed release formulations, extended release formulations, pulsatile release formulations, and mixed immediate release and controlled release formulations; or (d) any combination of (a), (b), and (c).

21. A method of treating a subject in need by administering to the subject an effective amount of nanostructured Olmesartan or its pharmaceutically acceptable ester, preferable Olmesartan Medoxomil, or co-crystal of claims 1-19.

22. Use of nanostructured Olmesartan or its pharmaceutically acceptable ester, preferable Olmesartan Medoxomil, or co-crystal of claims 1-19 for preparation of a medicament.

23. Use of nanostructured Olmesartan or its pharmaceutically acceptable ester, preferable Olmesartan Medoxomil, or co-crystal of claims 1-19 with a solubility at least about 0.07 mg/ml in water for decreasing the dosage used in the treatment of hypertension.

24. Use of nanostructured Olmesartan or its pharmaceutically acceptable ester, preferable Olmesartan Medoxomil, or co-crystal of claims 1-19 having instantaneous redispersibility in physiological mediums in the treatment of hypertension.

25. Use of nanostructured Olmesartan or its pharmaceutically acceptable ester, preferable Olmesartan Medoxomil, or co-crystal of claims 1-19 having reduced food and side effect in decreased dosage in the treatment of hypertension.

26. Use of nanostructured Olmesartan or its pharmaceutically acceptable ester, preferable Olmesartan Medoxomil, or co-crystal of claims 1-19 having increased absorption in human gastrointestinal tract for decreasing the dosage used in the treatment of hypertension.

27. Use of nanostructured Olmesartan or its pharmaceutically acceptable ester, preferable Olmesartan Medoxomil, or co-crystal of claims 1-19 having faster onset of action in decreased dosage in the treatment of hypertension.

28. Use of nanostructured Olmesartan or its pharmaceutically acceptable ester, preferable Olmesartan Medoxomil, or co-crystal of claims 1-19 having decreased variability in decreased dosage in the treatment of hypertension.
